(19) United States
(12) Patent Application Publication
(10) Pub. No.: US 2022/0392622 A1
(43) Pub. Date: Dec. 8, 2022

(54) System and Method for Improved Medical Contact Center

(71) Applicant: American Health Connection, Inc., Beverly Hills, CA (US)

(72) Inventor: Yuriy Kotiyar, Beverly Hills, CA (US)

(73) Assignee: American Health Connection, Inc., Beverly Hills, CA (US)

(21) Appl. No.: 17/745,866

(22) Filed: May 16, 2022

Related U.S. Application Data
(60) Provisional application No. 63/188,946, filed on May 14, 2021.

(51) Int. Cl. G16H 40/20; G16H 10/60

(52) U.S. Cl. CPC G16H 40/20; G16H 10/60

(57) ABSTRACT

Aspects of the present disclosure relate to medical contact centers that facilitate and prioritize continuity in patient care regardless of a patient's geographical location or the location of their medical provider and/or insurance company. Accordingly, a medical contact center capable of providing routing, real-time reporting, and patient interaction recording services utilizing multiple communication channels is described in accordance with examples of the present disclosure. While offering multiple communication channels to a patient or potential patient that may include, but are not limited to voice, email, web chat, and short messaging session (SMS) communications, the highest HIPAA and HITECH security standards may be practiced, ensuring a safe and secure environment for patients and their providers.

### CROSS REFERENCE TO RELATED APPLICATION

This application claims the benefit of U.S. Application Ser. No. 63/188,946, filed on May 14, 2021, which is incorporated by reference in its entirety.

### COPYRIGHT NOTICE

Contained herein is material that is subject to copyright protection. The copyright owner has no objection to the facsimile reproduction of the patent disclosure by any person as it appears in the Patent and Trademark Office patent files or records, but otherwise reserves all rights to the copyright whatsoever. Copyright © 2022 American Health Connection, Inc.

### FIELD OF THE INVENTION

Embodiments of the present invention generally relate to contact center management systems. More particularly, embodiments of the present invention relate to techniques for integrating contact center management systems with health care medical data.

### BACKGROUND

When a patient desires to contact a physician or medical provider regarding an appointment, medical procedure, or the like, the patient may often reach out to the medical provider directly or the office of the medical provider. While a medical provider may rely on a staff of individuals to field incoming requests from patients and potential patients; oftentimes, in anticipation of or following an appointment with the medical provider and/or in anticipation of or following a medical procedure, patients may be directed to contact one or more other resources, such as but not limited to an insurance company, another physician or medical provider, a hospital, physical therapy, or the like, as part of a care coordination process to ensure a medical procedure goes as smoothly as possible. However, as more and more resources and parties get involve, it is often difficult to ensure continuity in patient care and ensure that a patient is receiving the highest standard of care as efficiently as possible throughout the entire process of scheduling a procedure, having the procedure, and post procedure follow-up actions. Accordingly, patients may be required to check back in with a medical facility, such as a hospital or the like, for subsequent care or may miss vital information. Accordingly, there is a need to provide a healthcare network that allows patients to schedule healthcare anywhere that is accessible by a communications network so that the patient may receive high quality care regardless of their geographical location or the location of their health provider and/or insurance company.

### SUMMARY

The present disclosure provides medical contact centers that facilitate and prioritize continuity in patient care regardless of their geographical location or the location of their health provider and/or insurance company.

An objective of the disclosure is to provide apparatuses, systems, and methods to offer multiple communication channels to a patient or potential patient that may include, but are not limited to voice, email, web chat, and short messaging session (SMS) communications, while providing the highest HIPAA and HITECH security standards; thus, ensuring a safe and secure environment for patients and their providers.

Another objective of the disclosure is to reduce a medical provider's information technology (IT) costs and ensure rapid service. For example, custom interfaces, including bidirectional HL7 and SOA web services, or legacy file transfer interfaces, provide inter-operable system-to-system communications to facilitate real-time exchange of appointment and other healthcare data.

In some embodiments, artificial intelligence may be leveraged to recognize callers and ensure reminders are sent to aid in rescheduling and connect patients with a live agent if necessary. In examples, electronic health records (EHR) and electronic medical records (EMR) may be integrated with the medical contact center and offer bidirectional automated updates. In addition, various architectures and frameworks associated with EHR and EMR may be utilized, including but not limited to HL7 and FIHR.

Another objective of the disclosure is to provide answering services for medical providers utilizing experienced patient communication specialists (“agents”) that replace obsolete and traditional answering services. The agents may schedule appointments, re-schedule appointments, page a physician or medical provider, and handle other types of messages, such as asynchronous communication events (e.g., emails, texts, chats, etc.).

Another objective of the disclosure is to provide a physician, or medical provider a referral service. Accordingly, agents may follow client defined and preset guidelines and have the ability to search database for medical providers meeting the patient's and physician's requirements.

A further objective of the present disclosure it to facilitate the ability for an agent to conduct pre-authorizations for insurance coverage; thereby, permitting tracking pre-authorizations and authorizations in order to ensure that denials of claims are minimized.

Another objective of the disclosure is to provide various discharge follow-up options including identifying and calling high-risk patients within a satisfactory period of time. Such follow-up significantly reduces the rate of re-admissions by a percent much improved from the industry average.

These and other advantages will be apparent from the disclosure(s) contained herein. The above-described embodiments, objectives, and configurations are neither complete nor exhaustive. The Summary is neither intended nor should it be construed as being representative of the full extent and scope of the disclosure. Moreover, references made herein to “the disclosure” or aspects thereof should be understood to mean certain embodiments of the disclosure and should not necessarily be construed as limiting all embodiments to a particular description. The disclosure is set forth in various levels of detail in the Summary as well as in the attached drawings and Detailed Description and no limitation as to the scope of the disclosure is intended by either the inclusion or non-inclusion of elements, components, etc. in this Summary. Additional aspects of the disclosure will become more readily apparent from the Detailed Description particularly when taken together with the drawings.

### BRIEF DESCRIPTION OF THE DRAWINGS

The accompanying drawings, which are incorporated in and constitute a part of the specification, illustrate embodiments of the disclosure and together with the general description of the disclosure given above and the detailed description of the drawings given below, serve to explain the principles of the disclosures. Non-limiting and non-exhaustive examples are described with reference to the following Figures.

FIG. 1 is a block diagram of a communication system in accordance with examples of the present disclosure;

FIG. 2 depicts additional details of a communication system in accordance with examples of the present disclosure;

FIG. 3 is a block diagram depicting data structures used in accordance with examples of the present disclosure;

FIG. 4 depicts an example of a user interface in accordance with examples of the present disclosure;

FIG. 5 depicts a flow diagram depicting a method in accordance with examples of the present disclosure;

FIG. 6 depicts a second flow diagram depicting a second method in accordance with examples of the present disclosure; and

FIG. 7 depicts a block diagram illustrating physical components of a computing system in accordance with examples of the present disclosure.

It should be understood that the drawings are not necessarily to scale, and various dimensions may be altered. In certain instances, details that are not necessary for an understanding of the disclosure or that render other details difficult to perceive may have been omitted. It should be understood, of course, that the disclosure is not necessarily limited to the particular embodiments illustrated herein.

### DETAILED DESCRIPTION

In the following detailed description, references are made to the accompanying drawings that form a part hereof, and in which are shown by way of illustrations specific embodiments or examples. These aspects may be combined, other aspects may be utilized, and structural changes may be made without departing from the present disclosure. Embodiments may be practiced as methods, systems, or devices. Accordingly, embodiments may take the form of a hardware implementation, an entirely software implementation, or an implementation combining software and hardware aspects. The ensuing description provides embodiments and examples only and is not intended to limit the scope, applicability, or configuration of the claims. Rather, the ensuing description will provide those skilled in the art with an enabling description for implementing the embodiments and examples. It will be understood that various changes may be made in the function and arrangement of elements without departing from the spirit and scope of the appended claims.

Any reference in the description comprising an element number, without a sub-element identifier when a sub-element identifier exists in the figures, when used in the plural, is intended to reference any two or more elements with a like element number. When such a reference is made in the singular form, it is intended to reference one of the elements with the like element number without limitation to a specific one of the elements. Any explicit usage herein to the contrary or providing further qualification or identification shall take precedence.

The exemplary systems and methods of this disclosure will also be described in relation to analysis software, modules, and associated analysis hardware. However, to avoid unnecessarily obscuring the present disclosure, the following description omits well-known structures, components, and devices, which may be omitted from or shown in a simplified form in the figures or otherwise summarized.

For purposes of explanation, numerous details are set forth in order to provide a thorough understanding of the present disclosure. It should be appreciated, however, that the present disclosure may be practiced in a variety of ways beyond the specific details set forth herein.

When a patient desires to contact a physician or medical provider regarding an appointment, medical procedure, or the like, the patient may often reach out to the medical provider directly or the office of the medical provider. While a medical provider may rely on a staff of individuals to field incoming requests from patients and potential patients; oftentimes, in anticipation of or following an appointment with the medical provider and/or in anticipation of or following a medical procedure, patients may be directed to contact one or more other resources, such as but not limited to an insurance company, another physician or medical provider, a hospital, physical therapy, or the like, as part of a care coordination process to ensure a medical procedure goes as smoothly as possible. However, as more and more resources and parties get involved, it is often difficult to ensure continuity in patient care and ensure that a patient is receiving the highest standard of care as efficiently as possible throughout the entire process of scheduling a procedure, having the procedure, and post procedure follow-up actions. Accordingly, patients may be required to check back in with a medical facility, such as a hospital or the like, for subsequent care or may miss vital information.

Examples of the present disclosure are directed to medical contact centers that facilitate and prioritize continuity in patient care regardless of their geographical location or the location of their health provider and/or insurance company. Accordingly, a medical contact center capable of providing routing, real-time reporting, and patient interaction recording services utilizing multiple communication channels is described in accordance with examples of the present disclosure. While offering multiple communication channels to a patient or potential patient that may include, but are not limited to voice, email, web chat, and short messaging session (SMS) communications, the highest HIPAA and HITECH security standards may be practiced, ensuring a safe and secure environment for patients and their providers. In examples, a medical contact center may service multiple physicians and/or medical providers. To reduce a medical provider's information technology (IT) costs and ensure rapid service, examples of the present disclosure are directed to specialized interfaces to match each medical provider's IT configuration. For example, custom interfaces, including bidirectional HL7 and SOA web services, or legacy file transfer interfaces, provide inter-operable system-to-system communications to facilitate real-time exchange of appointment and other healthcare data. In addition, medical provider specific resource agents may handle every facet of a scheduling process, streamlining patient scheduling with a one call solution and providing full patient care coordination services that may include, but is not limited to pre-registration services, insurance verification services, customized language services, and follow-up services. In examples, patients are connected directly to a dedicated Patient Communication Specialists (PCS) that integrate custom voice, text, and email reminders based on appointment types, locations, procedures, and tests. In addition, artificial intelligence may be leveraged to recognize callers and ensure reminders are sent to aid in rescheduling and connect patients with a live agent if necessary. In examples, electronic health records (EHR) and electronic medical records (EMR) may be integrated with the medical contact center and offer bidirectional automated updates. In addition, various architectures and frameworks associated with EHR and EMR may be utilized, including but not limited to HL7 and FIHR.

In accordance with examples of the present disclosure, the medical contact center may also provide answering services for medical providers. That is, agents of the medical contact center may be experienced patient communication specialists and replace obsolete and traditional answering services. The agents may schedule appointments, re-schedule appointments, page a physician or medical provider, and handle other types of messages, such as asynchronous communication events (e.g., emails, texts, chats, etc.). Thus, patient no-show rates may be reduced, as traditional answering services are limited to the physician practice's ability to reschedule calls after hours. Coupled with appointment reminder service, patients have the ability to reschedule appointments 24/7 during a reminder call and reschedule their appointment in one call.

In accordance with examples of the present disclosure, the medical contact center may also provide a physician, or medical provider referral service. Accordingly, agents may follow client defined and preset guidelines and have the ability to search database for medical providers meeting the patient's and physician's requirements. For example, such requirements may include, but are not limited to a physician's age, sex, languages spoken, specialties, number of years in practice, office location, and hours of operation. Many times, an agent will be able to schedule an appointment during the first contact or call with the agent at the medical contact center. The medical contact center may also facilitate the ability for an agent to conduct pre-authorizations; tracking pre-authorizations and authorizations ensures that denials are minimized. In examples, the medical contact center may facilitate the agent to retrieve clinical data directly from an EMR. The agent may also directly contact an insurance company, provide the required forms be it by fax, email, and/or web submissions for examples, verify insurance prior to testing and procedure dates, notify patients of deductibles, co-pays, and co-insurances. Accordingly, utilizing a comprehensive process ensures that denials are an uncommon occurrence.

In accordance with examples of the present disclosure, agents associated with the medical contact center may provide various discharge follow-up options including identifying and calling high-risk patients within a satisfactory period of time, such as but not limited to twelve and/or twenty-four hours. Such follow-up significantly reduces the rate of re-admissions by as much as 40%, where the industry average is one in five patients requiring readmission within thirty days of discharge. Accordingly, medical provider resources associated with readmissions can be reduced. In examples, the agent may be specific to a medical provider. Further, such agents may pinpoint the most appropriate resource to address patient and physician treatment protocols, scheduling follow-up appointments, collecting data and reporting results to and from the patients and other resources, such as a hospital or other medical provider.

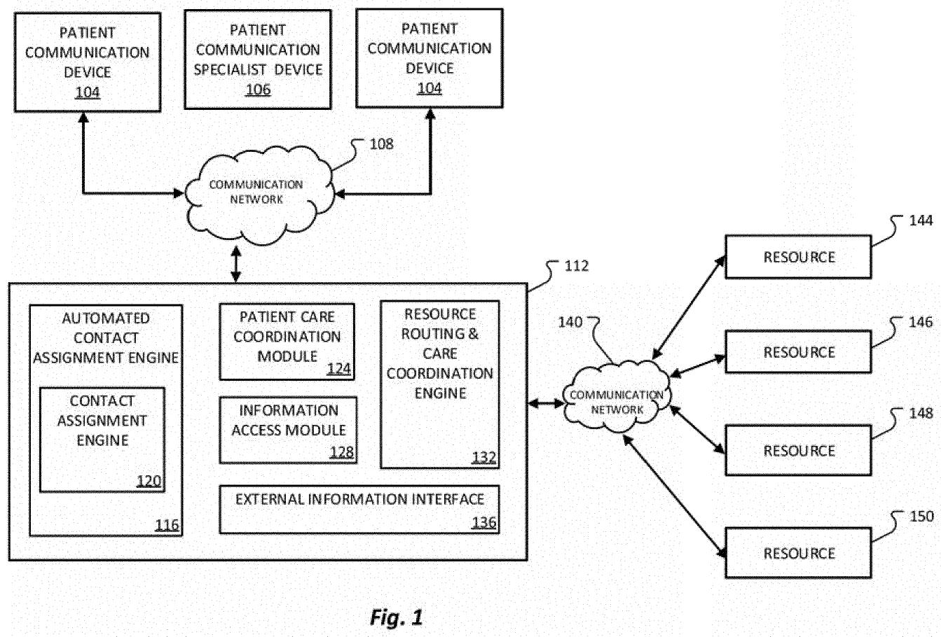

FIG. 1 depicts a communication system 100 including the medical contact center 112 in accordance with at least some examples of the present disclosure. The communication system 100 may be a distributed system and, in some examples, includes a communication network 108 connecting one or more patient communication devices 104 and patient care specialist devices 106 to a medical contact center 112. The medical contact center 112 may route one or more of the patient communication devices 104 to one or more resources 144-150, where the one or more resources 144-150 may be located remotely from one another and from the medical contact center 112. In examples, an automated contact assignment engine 116 may receive an initial contact from one or more of the patient communication devices 104, process the initial contact from the one or more patient communication devices 104 into a patient care work item, and assign the patient care work item to one or more of the resources 144-150. The patient care coordination module 124 may consider medical records, insurance, availability of resources, and a profile of the patient and/or physician when routing the patient care work item. In examples, a patient may contact the medical contact center 112 utilizing one of a variety of methods, or channels of communication. For example, a patient may utilize the patient communication device 104 to dial a toll-free number, initiate a chat session, initiate a two-way communication video call, and/or other synchronous and asynchronous methods of communication, to schedule a medical visit, schedule a medical procedure, or simply pose a question. As another non-limiting example, a patient may send an email to the medical contact center 112 with a request to schedule a medical visit, schedule a medical procedure, or simply pose a question.

The medical contact center 112 may receive the request and/or initiation of the communication session, determine an identity or identifier associated with the patient, and then generate a patient care work item. The patient care work item may then be assigned to a resource 144-150 via the patient care assignment engine and the resource routing and care coordination engine 132. In examples, the contact assignment engine 120 may retrieve medical records, resource schedules, and patient and/or physician profiles to determine or otherwise identify which resource 144-150 to assign the patient care work item. In examples, the patient content assignment engine may gather, or otherwise retrieve, information specific to the patient based on the patient care work item and information added to the patient care work item. For example, an identity or identifier associated with the patient may be used to retrieve a medical record of the patient; the medical record may be attached to, referenced, or otherwise included as part of the patient care work item. Based on the medical record, other information and/or resources, may be retrieved. For example, the medical record associated with the patient identity and/or patient identifier may indicate that a patient has previously initiated contact with three physicians or medical providers; alternatively, or in addition the medical record associated with the patient identity and/or patient identifier may indicate that a patient is associated with three physicians or medical providers—either as a primary care physician for example, or as a specialist. Accordingly, the contact assignment engine 120 may retrieve medical provider information for each of the three physicians or medical providers; the medical provider information may include but not limited to a physician or medical provider profile, a physician or medical provider schedule, and/or other resources generally associated with physicians and medical providers. Such information may then be included with, attached, referenced, or otherwise associated with the patient care work item.

In examples, the patient care assignment engine may access medical records, resource information, and/or medical provider information that is located within, maintained by, or otherwise managed by the medical contact center 112. Alternatively, or in addition, the medical contact center 112 may access information that may reside externally to the medical contact center 112. For example, the contact assignment engine 120 may request medical records associated with a patient identity or patient identifier; such a request may be sent to the information access module 128. The information access module 128, may determine that the medical records associated with the patient identifier or patient identity are not available locally at the medical contact center 112; accordingly, the medical contact center 112 may utilize an external information interface 136 to request, fetch, synchronize, or otherwise retrieve such medical records. In some examples, the medical contact center 112 may request authorization from a patient in connection with the request for medical record information. In examples, the medical record may be an EHR, EMR, patient health record, or other record including information associated with a patient. Various architectures and frameworks associated with EHR and EMR may be supported by the external information interface 136, including but not limited to HL7 and FIHR.

As another example, the contact assignment engine 120 may utilize the external information interface 136 to access insurance information and/or other resource information. For example, the information access module 128, may determine that insurance information associated with the patient identifier or patient identity is not available locally at the medical contact center 112; accordingly, the medical contact center 112 may utilize the external information interface 136 to request, fetch, synchronize, or otherwise retrieve such insurance information. In examples, the insurance information may include, but is not limited to, covered and non-covered procedures, deductible limits, co-pays, and/or expected out-of-pocket expenses associated with an expected procedure.

In examples, the contact assignment engine 120, based on the patient care work item, may assign the patient care work item to a resource 144-150; in examples, the resources 144-150 are available via the communication network 140, which may be the same as or similar to the communication network 108 previously described. The resource routing & care coordination engine 132 may handle the transfer of the patient care work item from within the medical contact center 112 to the one or more resources 144-150 assigned by the contact assignment engine 120. In examples, a resource 144-150 may interact with the capabilities provided by the medical contact center 112 to facilitate scheduling, post-care follow-up and other care coordination services.

In accordance with at least some examples of the present disclosure, the communication network 108 may comprise any type of known communication medium or collection of communication media and may use any type of protocols to transport messages between endpoints. The communication network 108 may include wired and/or wireless communication technologies. The Internet is an example of the communication network 108 that constitutes an Internet Protocol (IP) network consisting of many computers, computing networks, and other communication devices located all over the world, which are connected through many telephone systems and other means. Other examples of the communication network 108 include, without limitation, a standard Plain Old Telephone System (POTS), an Integrated Services Digital Network (ISDN), the Public Switched Telephone Network (PSTN), a Local Area Network (LAN), a Wide Area Network (WAN), a Session Initiation Protocol (SIP) network, a Voice over Internet Protocol (VoIP) network, a cellular network, and any other type of packet-switched or circuit-switched network known in the art. In addition, it can be appreciated that the communication network 108 need not be limited to any one network type, and instead may be comprised of a number of different networks and/or network types. Moreover, the communication network 108 may comprise a number of different communication media such as coaxial cable, copper cable/wire, fiber-optic cable, antennas for transmitting/receiving wireless messages, and combinations thereof. The communication network 140 may be the same as or similar to the communication network 108.

The patient communication devices 104 may correspond to customer communication devices. In accordance with at least some examples of the present disclosure, a patient may utilize their patient communication device 104 to initiate a patient care work item. Exemplary patient care work items may be initiated as a contact directed toward and received at the medical contact center 112, a web page request directed toward and received at a server farm (e.g., collection of servers) associated with the medical contact center 112, an application request (e.g., a request for application resources located on a remote application server at the medical contact center 112, such as a SIP application server), and the like. The patient care work item may be in the form of a message or collection of messages transmitted over the communication network 108 and in examples, may change over time to include additional information associated with the patient. For example, the work item may be transmitted as a telephone call, a packet or collection of packets (e.g., IP packets transmitted over an IP network), an email message, an Instant Message, an SMS message, a fax, and combinations thereof.

In accordance with at least some examples of the present disclosure, the patient communication devices 104 may comprise any type of known communication equipment or collection of communication equipment. Examples of a patient communication device 104 include, but are not limited to, a personal computer, laptop, Personal Digital Assistant (PDA), cellular phone, smart phone, telephone, tablet, mobile computer, or combinations thereof. In general, each patient communication device 104 may be adapted to support video, audio, text, and/or data communications with other patient communication devices 104 as well as the medical contact center 112. The type of medium used by the patient communication device 104 to communicate with other patient communication devices 104 may depend upon the communication applications available on the patient communication device 104.

In accordance with at least some examples of the present disclosure, the patient care work item is sent toward or otherwise made available to the resources 144-150 via the combined efforts of the contact assignment engine 120, the automated contact assignment engine 116, and the resource routing & care coordination engine 132. The resources 144-150 can either be completely automated resources (e.g., Interactive Voice Response (IVR) units, processors, servers, or the like), dedicated patient communication specialists utilizing resource communication devices (e.g., human PCSs utilizing a computer, telephone, laptop, or other device 106 etc.), or any other resource known to be used in contact centers, medical contact centers, and the like and may include other contact and contact centers.

The contact assignment engine 120 may reside in the automated contact assignment engine 116 or in a number of different servers or processing devices. In some examples, cloud-based computing architectures can be employed whereby one or more components of the automated contact assignment engine 116 are made available in a cloud or network such that they can be shared resources among a plurality of different users.

In some examples, the contact assignment engine 120 may dynamically assemble a team of resources 144-150 based on a patient care work item. Assembling a team of resources 1440-150 may include determining a combination of resources 144-150 having one or more attributes that match information associated with a patient care work item. Additionally, or in addition, assembling a team of resources 144-150 may include reserving a number of resources 144-150 associated with the medical contact center 112 for a specific amount of time and/or an expected involvement with the patient care work item and/or the team. This specific amount of time and/or the expected involvement may be based, at least partially, on historical information associated with one or more of prior patients and/or specific patient care work item types. The automated contact assignment engine 116 may include a Workforce Management system that matches PCS skills and work schedules to projected volumes and types of calls. The automated contact assignment engine 116 may include a built-in Quality Control and Auditing module that allows Auditors to efficiently monitor and manage quality based on parameters defined for each healthcare facility.

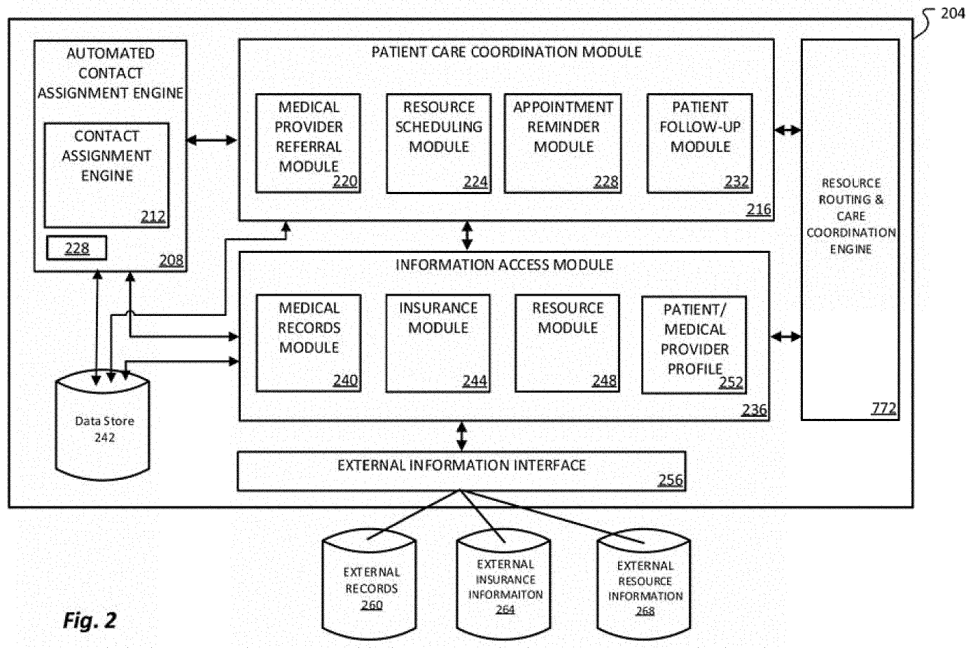

FIG. 2 provides additional examples directed to the medical contact center 204. The medical contact center 204 may be the same as or similar to the medical contact center 112 previously described. The medical contact center 204 may include the automated contact assignment engine 208 and the contact assignment engine 212. The automated contact assignment engine 208 may be the same as or similar to the automated contact assignment engine 116 previously described. The contact assignment engine 212 may be the same as or similar to the contact assignment engine 120 as previously described. An initiation of a communication may be received at the automated contact assignment engine 208; automated contact assignment engine 208 may consume the communication and generate or otherwise update a patient care work item. The patient care work item may be associated with an identity of the patient that initiated the communication and/or an identifier associated with the patient that initiated the communication.

In examples, the automated contact assignment engine 208 may determine a reason for the patient communication. For example, the reason for the communication may be associated with a request for an appointment or medical procedure. In examples, the reason for the patient communication may be determined from an interactive voice response unit, a web page submission, a parsing of an email, or entry by an agent or dedicated patient communication specialists, or the like. Such information may be associated with the patient in a patient profile 252. In some examples, an initial resource, such as resource 144, may obtain the reason for the patient call and obtain other information about the patient to generate a patient profile 252. The initial resource 144 may correspond to an agent and/or a dedicated patient communication specialist for example. In examples, the automated contact assignment engine 208 has the ability to manage outbound health management campaigns such as Annual Wellness visits and Chronic Care Management processes.

In instances where a patient may be calling or otherwise contacting the medical contact center 204 for a first time, the patient care assignment engine may make use of the patient care coordination module 216 to identify a medical provider or physician using the medical provider referral module 220, schedule a physician or medical provider using the resource scheduling module 224, and generate a reminder and/or follow-up communications based on the scheduled appointment using the appointment reminder module 228 and/or the patient follow-up module 232. Further, a user profile may be generated and stored via the information access module 236; the patient profile may be stored as a patient/medical provider profile 252.

In examples, the patient may already have an existing patient profile located at the patient/medical provider profile 252 and may be calling to schedule another appointment or other procedure; accordingly, based on a reason for the patient call, a physician or medical provider may be identified utilizing the medical provider referral module 220. An agent, such as the dedicated patient communication specialist may access one or more resources associated with the medical provider using the resource scheduling module 224.

In examples, the resource scheduling module 224 may utilize the information access module 236 to view, pull, or request a schedule associated with a medical provider, such as a physician. Accordingly, an agent may book an appointment, medical procedure, or test for example, utilizing the resource scheduling module 224. In addition, the resource scheduling module 224 may access, view, pull, or request post medical procedure resource schedules. For example, a medication service may be accessed to fulfill a patient's subscription; transportation services may be accessed to arrange for patient transportation to and from the medical provider; physical therapy services may be scheduled, and/or other follow-up appointments may be scheduled. In some examples, medical procedure resources may be scheduled as well, such as but not including an exam room, an operating room, a specialized staff, medical equipment, or other resources used before, during, and/or after a medical procedure. Thus, a schedule for each resource may be accessed via one or more modules of the information access module 236 and more specifically, one or more resource modules configured to interact with and/or exchange information with one or more resources.

In examples, the modules of the information access module 236 may interact with external sources of information utilizing the external information interface 256. For example, the resource module 248 may interact with external sources of information (e.g., resource schedules, transportation services, etc.) utilizing the external information interface 256. As another example, the medical records module 240 may interact with the external information interface to interface with an external records repository, framework, and/or architecture 260 to obtain records, such as but not limited to EHRs, MHRs, and PHRs. In examples, the external information interface 256 may interact with HL7 and FIHR frameworks and architectures and stored via the medical records module 240 and associated with the patient/medical provider profile 252.

As another example, the insurance module 244 may interact with the external information interface 256 to access external information 264. Insurance information accessed and/or retrieved by the external information interface 256 may be stored in or otherwise associated with the patient profile 252 and/or an insurance module 244. In examples, the insurance information may be utilized to conduct insurance pre-authorizations.

In examples, the contact assignment engine 212 may access the one or more modules of the information access module and/or one and a patient/medical provider profile 252 when assigning the patient care work item to a resource. In examples, the patient care assignment engine may add information to a patient care work item and then determine a resource based on the information. As an example, the patient care work item may include information identifying a medical provider; accordingly, a medical provider profile, from the patient/medical provider profile 252 may be obtained. In some instances, the medical care provider profile may indicate or otherwise identify a resource. The contact assignment engine 212 may then assign the patient care work item to the identified resource and reply on the resource routing & care coordination engine to transfer the patient care work item to the selected resource. In examples, transferrin the patient care work item to the identified or selected resource may refer to routing a communication medium to the resource. For example, a contact by a patient via a phone call may be routed or transferred to a selected resource via the same or different communication mechanism (e.g., a phone call, chat, email, voicemail etc.). In addition, the patient care work item may be updated to reflect the selected resource.

As previously discussed, each of the modules of the patient care coordination module 216 and the information access module 236 may be accessible by one or more resources. As an example, agents may follow client defined and preset guidelines and have the ability to search a database for medical providers meeting a patient's and medical care provider's requirements. That is, a patient may initiate contact with the medical contact center 204 and a patient care work item may be created. The patient care work item may indicate that the patient is calling to schedule a procedure with one or more medical providers, some of which being new to the patient. Accordingly, an agent or dedicated patient communication specialist may utilize the medical provider referral module 220 to locate a medical provider. The medical provider referral module 220 may access the patient/medical provider profile 252, where such profile may include medical provider information, guidelines, and requirements. Such information, guidelines, and requirements may include but are not limited a physician's age, sex, languages spoken, specialties, number of years in practice, office location, and hours of operation.

The agent, or dedicated patient communication specialists, may also utilize the resource scheduling module 224 to schedule an appointment during the call. The medical contact center may also facilitate the ability for an agent to conduct pre-authorizations using the insurance module 244. In addition, the agent, or medical contact center 204, may setup a reminder of the appointment using the appointment reminder module 228. Prior to the scheduled appointment, the appointment reminder module 228 may cause a reminder to be sent to the patient via one or more means of communication (e.g., phone, email, text, etc.). In some examples, the patient, having received the reminder may choose to reschedule the appointment; such rescheduling may occur via the appointment reminder module 228 and/or by initiating contact with the medical contact center 204 and being routed to an appropriate resource. In examples, the appointment reminder module 228 may be located in the automated contact assignment engine 208.

In some examples, a patient follow-up module 232 may determine one or more medical appointments, medical procedures, and/or medical tests that have been conducted and update and/or create a patient care work item to reflect such appointment, test, and/or procedure completion. Accordingly, a work item may be created and/or routed to resource, such as a dedicated patient communication specialist for follow-up. In some examples, the means of communication and the follow-up time frame (e.g., within twenty-four hours) may be determined by the procedure, the medical care provider guidelines, and/or as a default configured by the medical contact center 204. In some examples, the patient follow-up module 232 may be accessed by an agent or patient communication specialist to set the follow-up reminder. Alternatively, or in addition, the patient follow-up module may automatically set a follow-up reminder based on an indication that an appointment, test, and/or procedure occurred.

The data store 242 may store information and/or data associated with a patient care work item, patient/medical provider profile 252, scheduling information, and/or reminders and follow-ups. In examples, the contact assignment engine 208, the patient care coordination module 216, and the information access module 236 may access the data store 242.

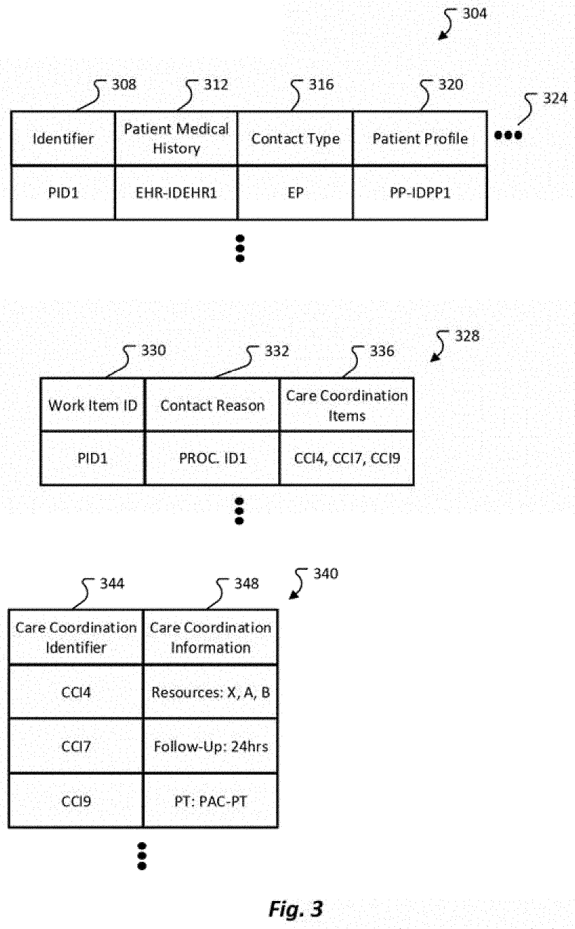

FIG. 3 depicts data structures associated with one or more patient care work items in accordance with examples of the present disclosure. A data structure 304 may be created when a patient contacts or initiate contacts with a medical contact center, such as the medical contact center 112 and/or 204. The data structure may be associated with a patient care work item or may be a patient care work item. The data structure 304 may include an identifier 308 identifying the entry or patient care work item; a reference to patient medical history 312 including but not limited to one or more medical records (e.g., EHR, MHR, etc.); a reference to a patient type 316 (e.g., new, existing); a reference to a patient profile 320 (e.g., a patient profile at the patient/medical provider profile 252). More or fewer fields may be present in the data structure 304. In addition, a data structure 328 may store a contact reason 332 associated with a patient care work item 330 and one or more care coordination items 336 that may associated with the contact reason. For example, the contact reason may refer to scheduling a procedure identified as PROC. PID1; coordination care items may be referenced as CCI4, CCI7, and CCI9. In addition, a data structure 340 may provide additional information for each coordination care item. For example, a coordination care item identified as CCI4 may refer to resources needed; another coordination care item, such as CCI7 may refer to a follow-up appointment time frame. The data structures of FIG. 3 may be stored in the data store 242.

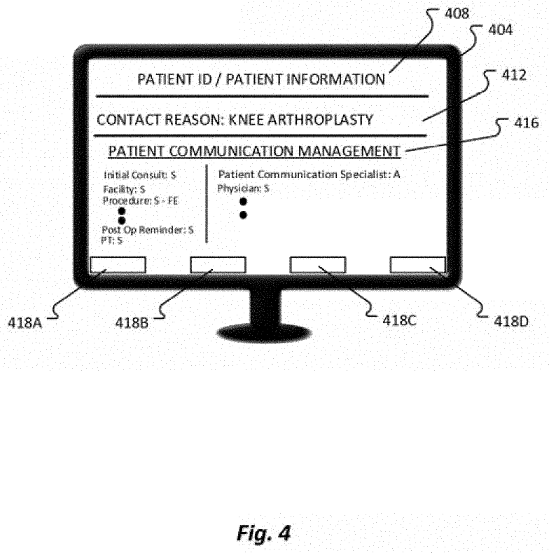

FIG. 4 depicts an example of a graphical user interface 404 utilized by an agent or dedicated patient communication specialist, in accordance with examples of the present disclosure. The graphical user interface 404 may include patient information 408, a contact reason 412, and patient care coordination information 416. Accordingly, as a patient communication specialist may be interacting with a patient to schedule an appointment, procedure, or test, the graphical user interface 404 may indicate which care coordination items are to be scheduled and/or completed. Information populating the graphical user interface 404 may be retrieved or otherwise accessed from the data store 242 for example. In addition, the information displayed at the graphical user interface 404 may represent information in one or more of the data structures discussed in FIG. 3. In some examples, the information may be provided by trained patient communication specialists according to one or more medical provider guidelines. In examples, the graphical user interface 404 includes one or more areas 418 that integrate call/communication controls such as call statistics, hold, transfer, disconnect, status.

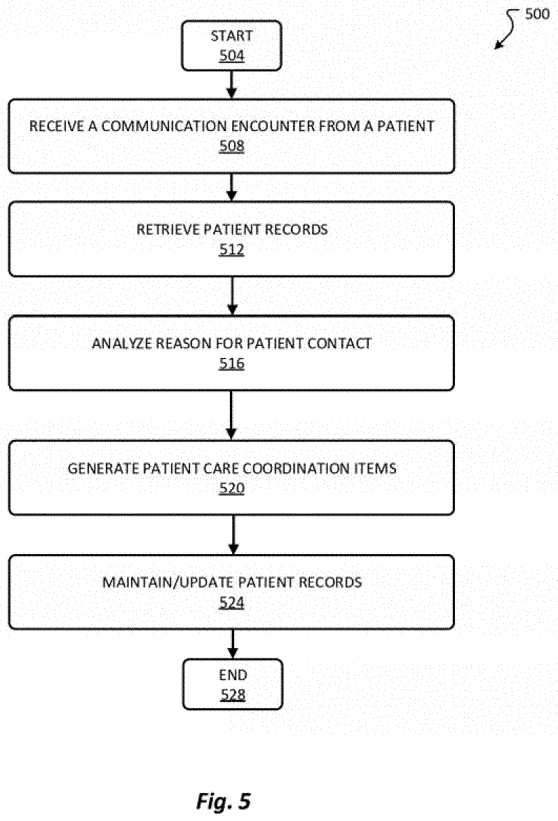

FIG. 5 depicts details of a method 500 for establishing patient care coordination items in accordance with examples of the present disclosure. A general order for the steps of the method 500 is shown in FIG. 5. Generally, the method 500 starts at 504 and ends at 528. The method 500 may include more or fewer steps or may arrange the order of the steps differently than those shown in FIG. 5. The method 500 can be executed as a set of computer-executable instructions executed by a computer system and encoded or stored on a computer readable medium. In examples, aspects of the method 500 are performed by one or more processing devices, such as a computer or server. Further, the method 500 can be performed by gates or circuits associated with a processor, Application Specific Integrated Circuit (ASIC), a field programmable gate array (FPGA), a system on chip (SOC), or other hardware device. Hereinafter, the method 500 shall be explained with reference to the systems, components, modules, software, data structures, user interfaces, etc. described in conjunction with FIGS. 1-4.

The method 500 starts at 504, where flow may proceed to 508. The method 500 can be executed as a set of computer-executable instructions executed by a computer system and encoded or stored on a computer readable medium. At 508, a communication encounter from a patient may be received at a medical contact center, such as the medical contact center 112 and/or 204. In examples, the contact may occur using any one of the previously described communication channels and a patient care work item may be generated for the contact. In examples, the method may proceed to 512 where one or more patient records are retrieved. As previously described, the one or more patient records may be identified from a patient identifier or identifier associated with the patient. In addition, a reason associated with the patient contact may be analyzed or otherwise ascertained at 516. In examples, an automated resource may initially identify the reason from a voice response or some other indication. Alternatively, or in addition, the medical records retrieved at 512 may indicate a likely reason for the call and such reason may be confirmed by the patient. In some examples, a previous patient care work item may indicate a reason for the contact and may be verified by the patient. In some examples, an agent or patient communication specialist may identify a reason for the contact.

The method may proceed to 520, where one or more patient care coordination items may be generated. For example, a reason for the call may be to schedule a medical procedure, such as knee arthroplasty. Based on the procedure, one or more care coordination items may be generated and/or identified at 520. In addition, such care coordination items may be rendered to a graphical user interface, such as the graphical user interface of 404. Accordingly, a patient communication specialist may schedule, complete, and/or verify each of the care coordination items. The patient record, such as a medical record and/or the patient care work item may be updated at 524. The method 500 may end at 528.

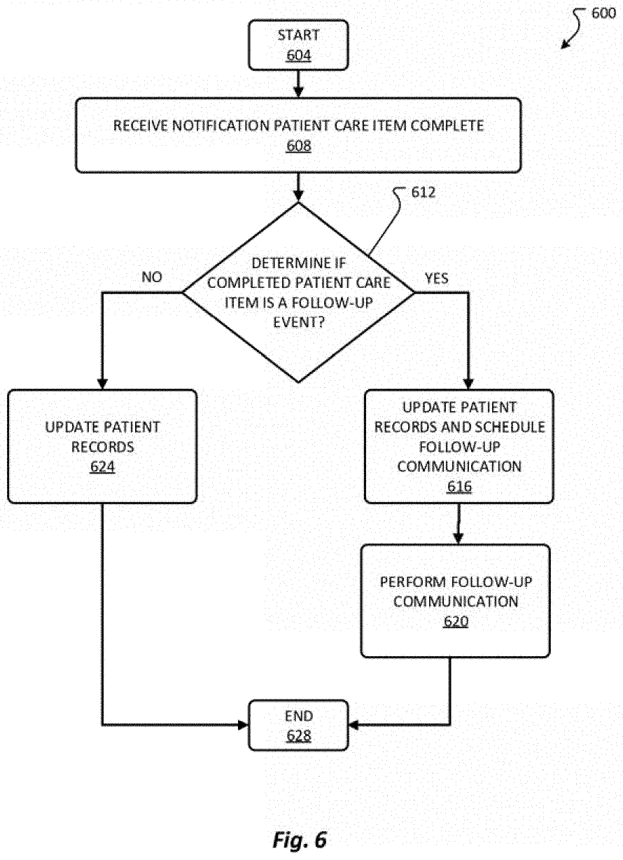

FIG. 6 depicts details of a method 600 for establishing a follow-up communication in accordance with examples of the present disclosure. A general order for the steps of the method 600 is shown in FIG. 6. Generally, the method 600 starts at 604 and ends at 628. The method 600 may include more or fewer steps or may arrange the order of the steps differently than those shown in FIG. 6. The method 600 can be executed as a set of computer-executable instructions executed by a computer system and encoded or stored on a computer readable medium. In examples, aspects of the method 600 are performed by one or more processing devices, such as a computer or server. Further, the method 600 can be performed by gates or circuits associated with a processor, Application Specific Integrated Circuit (ASIC), a field programmable gate array (FPGA), a system on chip (SOC), or other hardware device. Hereinafter, the method 600 shall be explained with reference to the systems, components, modules, software, data structures, user interfaces, etc. described in conjunction with FIGS. 1-5.

The method 600 starts at 604, where flow may proceed to 608. At 608, an indication or notification that a patient care item is complete or has been completed is received. For example, a patient care item may refer to a medical procedure, test, and/or appointment. Upon completion, for example a medical care provider indicating that the care coordination item is complete, a patient communication specialist indicating the care coordination is complete, and/or after a scheduled appointment, test, or procedure, a determination may be made at 612 if the patient care item is a follow-up event. For example, some patient care items, such as medical procedures, may be a follow-up event whereas other patient care items, such as medication refill, may not be a follow-up event. Accordingly, at 612, if the patient care item is a follow-up event, the method 600 may proceed to 616 where a follow-up communication is scheduled. Such follow-up communication may be recorded in a patient medical record and/or may be scheduled by the patient follow-up module 232 for example. In examples, and at the scheduled time, the method 600 may proceed to 620 where the follow-up communication is performed. For example, a phone call is placed to the patient and/or a text or email is sent to the patient. In examples where the patient care item is not a follow-up event at 612, the method 600 may proceed to 624 and update a patient records to indicate that the patient care item is complete. The method 600 may end at 628.

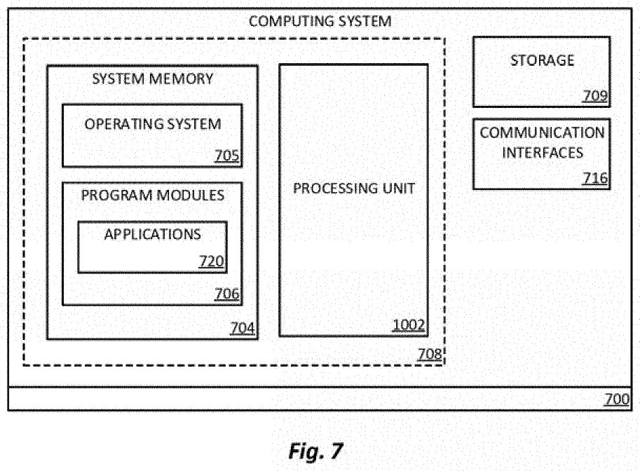

FIG. 7 is a block diagram illustrating physical components (e.g., hardware) of a computing system 700, such as a medical contact center, with which aspects of the disclosure may be practiced. The computing system 700 components described below may be suitable for the computing and/or processing devices described above. In a basic configuration, the computing system 700 may include at least one processing unit 702 and a system memory 7704. Depending on the configuration and type of computing device, the system memory 704 may comprise, but is not limited to, volatile storage (e.g., random-access memory (RAM)), non-volatile storage (e.g., read-only memory (ROM)), flash memory, or any combination of such memories.

The system memory 704 may include an operating system 705 and one or more program modules 706 suitable for running software application 720, such as one or more components supported by the systems described herein. As examples, system memory 704 may include patient care coordination module 124/216, the information access module 128/236, the resource routing & care coordination engine 132/272, the external information interface 136/256, and/or the automated contact assignment engine 116/208. The computing system 700 may have additional features or functionality. For example, the computing system 700 may also include additional data storage devices (removable and/or non-removable) such as, for example, magnetic disks, optical disks, or tape. Such additional storage is illustrated in FIG. 7 by a removable and non-removable storage 709.

As stated above, a number of program modules and data files may be stored in the system memory 704. While executing on the processing unit 702, the program modules 706 (e.g., patient care coordination module 124/216, the information access module 128/236, the resource routing & care coordination engine 132/272, the external information interface 136/256, and/or the automated contact assignment engine 116/208) may perform processes including, but not limited to, the aspects, as described herein. Other program modules that may be used in accordance with aspects of the present disclosure may include electronic mail and contacts applications, email, medical records, call logs, word processing applications, spreadsheet applications, database applications, slide presentation applications, drawing or computer-aided programs, etc.

Furthermore, embodiments of the disclosure may be practiced in an electrical circuit discrete electronic elements, packaged or integrated electronic chips containing logic gates, a circuit utilizing a microprocessor, or on a single chip containing electronic elements or microprocessors. For example, embodiments of the disclosure may be practiced via a system-on-a-chip (SOC) where each or many of the components illustrated in FIG. 7 may be integrated onto a single integrated circuit. Such an SOC device may include one or more processing units, graphics units, communications units, system virtualization units and various application functionality, all of which are integrated (or “burned”) onto the chip substrate as a single integrated circuit. When operating via an SOC, the functionality, described herein, with respect to the capability of client to switch protocols may be operated via application-specific logic integrated with other components of the computing system 700 on the single integrated circuit (chip). Embodiments of the disclosure may also be practiced using other technologies capable of performing logical operations such as, for example, AND, OR, and NOT, including but not limited to mechanical, optical, fluidic, and quantum technologies. In addition, embodiments of the disclosure may be practiced within a general-purpose computer or in any other circuits or systems.

The computing system 700 may also have one or more input device(s) such as a keyboard, a mouse, a pen, a sound or voice input device, a touch or swipe input device, etc. Output device(s) such as a display, speakers, a printer, etc. may also be included. The aforementioned devices are examples and others may be used. The computing system 700 may include one or more communication interface 716, allowing communications with other computing devices and information system. Examples of a communication interface 716 include, but are not limited to, radio frequency (RF) transmitter, receiver, and/or transceiver circuitry; universal serial bus (USB), parallel, and/or serial ports.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” “A, B, and/or C,” and “A, B, or C” means A alone, B alone, C alone, A and B together, A and C together, B and C together, or A, B and C together.

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

Aspects of the present disclosure may take the form of an embodiment that is entirely hardware, an embodiment that is entirely software (including firmware, resident software, micro-code, etc.) or an embodiment combining software and hardware aspects that may all generally be referred to herein as a “circuit,” “module,” or “system.” Any combination of one or more computer-readable medium(s) may be utilized. The computer-readable medium may be a computer-readable signal medium or a computer-readable storage medium.

A computer-readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer-readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

A computer-readable signal medium may include a propagated data signal with computer-readable program code embodied therein, for example, in baseband or as part of a carrier wave. Such a propagated signal may take any of a variety of forms, including, but not limited to, electro-magnetic, optical, or any suitable combination thereof. A computer-readable signal medium may be any computer-readable medium that is not a computer-readable storage medium and that can communicate, propagate, or transport a program for use by or in connection with an instruction execution system, apparatus, or device. Program code embodied on a computer-readable medium may be transmitted using any appropriate medium, including, but not limited to, wireless, wireline, optical fiber cable, RF, etc., or any suitable combination of the foregoing.

The terms “determine,” “calculate,” “compute,” and variations thereof, as used herein, are used interchangeably, and include any type of methodology, process, mathematical operation, or technique.

The term “means” as used herein shall be given its broadest possible interpretation in accordance with 35 U.S.C., Section 112(f) and/or Section 112, Paragraph 6. Accordingly, a claim incorporating the term “means” shall cover all structures, materials, or acts set forth herein, and all of the equivalents thereof. Further, the structures, materials or acts and the equivalents thereof shall include all those described in the summary, brief description of the drawings, detailed description, abstract, and claims themselves.

What is claimed is:

1. A method for communicating with one or more patients while satisfying HIPAA and HITECH security standards comprising:
receiving a communication from a patient;
retrieving one or more patient records based on the received communication;
analyzing one or more reasons associated with receiving the communication from the patient;
generating one or more patient care coordination items based on the analysis of the one or more reasons; and
updating the one or more patient records to indicate an association with the generated one or more patient care coordination items.

2. The method of claim 1, further comprising:
receiving a notification that the one or more patient care items are complete;
determining that the one or more patient care items require a follow-up event;
updating one or more patient care items associated with the one or more patient records based on the determination that the one or more patient care items require a follow-up event;
scheduling a follow-up communication specific to the one or more patient care items determined to require a follow-up event at a scheduled time; and
initiating a communication with the patient in accordance with the scheduled time, wherein the scheduled time is dependent upon the patient care item.

3. The method of claim 1, further comprising:
receiving a notification that a patient care item is complete; and determining that the patient care item does not require a follow-up event.

4. The method of claim 1, further comprising rendering the one or more patient care coordination items to a graphical user interface.

5. The method of claim 1, wherein analyzing the one or more reasons associated with receiving the communication from the patent is based at least in part on a previous patient care work item associated with the patient.

6. The method of claim 5, wherein analyzing the one or more reasons associated with receiving the communication from the patent is performed using artificial intelligence.

7. The method of claim 1, wherein receiving a communication from a patient further comprises utilizing a custom interface including one or more of a bidirectional HL7 or SOA web service.

8. The method of claim 1, further comprising utilizing a medical provider referral module to locate a medical provider associated with the one or more patient care coordination items.

9. The method of claim 8, wherein utilizing the medical provider referral module to locate the medical provider further comprises accessing a medical provider profile, wherein the medical provider profile includes at least one of medical provider information, guidelines, or requirements.

10. The method of claim 9, wherein the medical provider profile includes at least one of a physician's age, sex, languages spoken, specialties, number of years in practice, office location, and hours of operation.

11. A medical contact center comprising:
an information access module, wherein the information access module is communicably coupled to a computer-readable medium storing instructions which when executed by the information access module, causes the information access module to:
store a patient profile, and
store a medical provider profile; and
a patient care coordination module, wherein the patient care coordination module is communicably coupled to the information access module, and wherein the patient care coordination module is communicably coupled to a computer-readable medium storing instructions, which when executed by the patient care coordination module, causes the patient care coordination module to:
identify a medical provider, and
schedule a patient visit with the medical provider.

12. The medical contact center of claim 11, wherein the instructions, when executed by the patient care coordination module, cause the patient care coordination module to conduct an insurance pre-authorization check.

13. The medical contact center of claim 11, wherein the instructions, when executed by the patient care coordination module, causes the patient care coordination module to:
generate a follow-up communication; and
generate a reminder of the patient visit with the medical provider.

14. The medical contact center of claim 11, further comprising:
a contact assignment engine, wherein the contact assignment engine is communicably coupled to a computer-readable medium storing instructions, which when executed by the contact assignment engine, causes the contact assignment engine to:
receive a communication from a patient;
assign a resource to a patient care work item associated with the patient;
retrieve one or more patient records associated with the patient;
analyze one or more reasons associated with the communication from the patient;
generate one or more patient care coordination items based on the analysis of the one or more reasons;
update the patient profile; and
update the medical provider profile.

15. The medical contact center of claim 11, further comprising:
an external information interface, wherein the external information interface is communicably coupled to the information access module, wherein the external information interface is communicably coupled to a computer-readable medium storing instructions, which executed by the external information interface, causes the external information interface to access one or more of: external resource information, external records, or external insurance information.
